(19) United States
(12) Patent Application Publication
(10) Pub. No.: US 2022/0198831 A1
(43) Pub. Date: Jun. 23, 2022

(54) SYSTEM FOR DETERMINING ONE OR MORE CHARACTERISTICS OF A USER BASED ON AN IMAGE OF THEIR EYE USING AN AR/VR HEADSET

(71) Applicant: Delphinium Clinic Ltd., Dublin (IE)

(72) Inventors: Kate Coleman, Dublin (IE); Jason Coleman, Churchtown (IE)

(21) Appl. No.: 17/554,374

(22) Filed: Dec. 17, 2021

Related U.S. Application Data
(60) Provisional application No. 63/126,592, filed on Dec. 17, 2020.

(51) Int. Cl. G06V 40/19; G06T 7/00; G06T 7/11

(52) U.S. Cl. CPC G06V 40/19; A61B 3/12; G06T 7/11; G06T 7/0012

(57) ABSTRACT

A system for determining one or more characteristics of a user based on an image of their eye includes a headset having a camera configured to acquire an image of the user's eye, and a computing device communicatively coupled to the camera and configured to receive the image of the user's eye and determine one or more characteristics of the user based on the received image.

### CROSS REFERENCE TO RELATED APPLICATION

The present application claims the benefit of U.S. provisional application Ser. No. 63/126,592, filed Dec. 17, 2020, which is hereby incorporated by reference in its entirety.

### FIELD OF THE INVENTION

This invention relates to a system for determining one or more characteristics of a user based on an image of their eye.

### BACKGROUND TO THE INVENTION

Every eye has a unique Optic Nerve Head (ONH) and neighboring area features which change with age, at the earliest sign of ONH features disease, and as the surrounding adjacent area/retina/choroid/develops with myopia, other refractive errors or disease.

The area mentioned also changes before any obvious clinical change of the ONH is detectable in early ONH diseases such as glaucoma, or with silent haemorrhage on the ONH in diabetic retinopathy, for example. The size and features of the ONH area vary between different races and with refractive error. A young Asian myopic patient may have an ONH and surrounding area larger than a non-myopic Asian child of the same age, or a Caucasian adult. The area between the ONH and the surrounding retina changes as the eyeball ages and as myopia occurs. Unlike face or iris recognition, a live image of the ONH and retina is internal, and gaze evoked, so cannot be unknowingly captured or altered. This may be particularly useful regarding cyber safety and cybersecurity of vulnerable groups such as children.

The ONH and surrounding area is easy for an ophthalmologist to classify as that of a child or an adult. Computer Vision and Artificial Intelligence algorithms can perform the same classification on a 2D color image of the Optic Nerve Head (ONH) and surrounding region without any clinical expertise.

The ONH itself loses axon fibres as the eye ages. Deep neural networks can identify the age of a person using a 2D retinal photograph. The ONH is known to lose axons with degenerative conditions such as Alzheimer's disease.

### SUMMARY OF THE INVENTION

The present invention aims to provide a system which is able to quickly and easily determine one or more characteristics of a user, such as the age and/or eye health of the user, based on image analysis of the user's eyes using AI as mentioned above. Further, the system is configured to monitor the one or more characteristics of the user and alert the user to changes in one or more of the characteristics, such as may occur to the ONH and surrounding area, as happens for example with silent disease changes of the eye.

Embodiments of the present invention provide a system for determining one or more characteristics of a user based on an image of their eye, acquired using a headset such as an Augmented Reality (AR)/Virtual reality headset (VR) or any camera on a Head Mounted Set or spectacle type frame. The system including an AR/VR headset including a camera designed to capture and use the image of the optic nerve head (ONH) area, within a field of vision of plus or minus 45 degrees of the macula, in order to a) function as a biometric device and identify the wearer and b) to ascertain the age of the wearer using a platform of computer vision and artificial intelligence algorithms and c) to identify ONH/retina interface changes with refractive error changes, such as early myopia and d) to confirm the gender of the wearer.

Accordingly, aspects of the present invention provide a system for determining one or more characteristics of a user based on an image of their eye, the system including:
a headset having a camera configured to acquire an image of the user's eye;
a computing device, communicatively coupled to the camera, which is configured to:
   receive the image of the user's eye; and
   determine one or more characteristics of the user based on the received image.

Optionally, the headset includes:
a substantially helmet-like headset that is configured to encapsulate at least a portion of the user's head; or
the headset includes a pair of glasses.

For example, the one or more characteristics which are determined may include one or more of: the age of the user, identity of the user, gender of the user, one or more health characteristics of the user.

Optionally, the headset includes an augmented reality (AR) or virtual reality (VR) headset.

Preferably, the image of the user's eye includes an image of the user's retina, preferably of the optic nerve head, and optionally plus or minus the eye surface and surrounding eyelid structures.

Optionally, the image of the user's retina includes the Optic Nerve Head (ONH) and surrounding area, such as a 45 degree field surrounding the ONH.

The computing device may be further configured to provide the determined characteristics to the user.

Optionally, the headset includes a display which is configured to visually display the determined characteristics to the user.

The computing device may include a display which is configured to visually display the determined characteristics to the user.

Optionally, the computing device is configured to acquire a plurality of images of the user's eyes at predetermined intervals.

Optionally, the computing device is configured to compare the determined characteristics of the user across the plurality of images and alert the user to one or more changes in their determined characteristics over a period of time.

Optionally, the computing device is configured to monitor the determined characteristics of the user's eyes over a period of time to determine any changes in the determined characteristics which may be indicative of one or more diseases or the like.

Optionally, to determine the one or more characteristics of the user based on the received image the computing device is configured to:
segment the image of the user's eye into multiple segments each containing blood vessels and neuroretinal rim fibres;
extract features from the segmented images, the features describing relationships between the blood vessels themselves and between the blood vessels and the neuroretinal rim fibres in each of the segmented images; and
identify characteristics of the eye based on the extracted features.

Optionally, the computing device is configured to superimpose multiple concentric geometric patterns on the multiple segments. The concentric geometric patterns further segmenting the image of the user's eye and advantageously making it easier and quicker to determine identify features within the images.

Optionally, the geometric patterns are in the form of concentric circles, ellipses, squares, or triangles.

Optionally, the extracted features additionally or alternatively include elements of the eye which intersect with one or more concentric geometric patterns superimposed thereon.

Optionally, the computing device is further configured to classify the image of the eye based on the identified characteristics.

Optionally, to determine the one or more characteristics of the user based on the received image the computing device is configured to:
segment the image of the user's eye into multiple segments,
superimpose multiple concentric geometric patterns onto the multiple segments;
extract features from the segmented images, the features including elements of the eye which intersect with one or more concentric geometric patterns; and
identify characteristics of the eye based on the extracted features.

A second aspect of the present invention provides a method for determining one or more characteristics of a user based on an image of their eye, the method including:
providing a user with a headset including a camera;
acquiring an image of the user's eye using the camera;
transmitting the acquired image of the user's eye to a computing device which is communicatively coupled to the camera; and
determining one or more characteristics of the user based on the received image.

Optionally, determining one or more characteristics of the user based on the received image includes:
segmenting the image of the user's eye into multiple segments each containing blood vessels and neuroretinal rim fibres;
extracting features from the segmented images, the features describing relationships between the blood vessels themselves and between the blood vessels and the neuroretinal rim fibres in each of the segmented images; and
identifying characteristics of the eye based on the extracted features.

Optionally, the method further includes superimposing multiple concentric geometric patterns on the multiple segments.

Optionally, the geometric patterns are in the form of concentric circles, ellipses, squares, or triangles.

Optionally, the method further includes:
acquiring a plurality of images of the user's eyes at predetermined intervals;
comparing the determined characteristics of the user across the plurality of images; and
alerting the user to one or more changes in their determined characteristics over a period of time.

A third aspect of the present invention provides the use of a headset for determining one or more characteristics of a user based on an image of their eye using the method as provided in the second aspect of the invention.

These and other objects, advantages, purposes and features of the present invention will become apparent upon review of the following specification in conjunction with the drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

Embodiments of the present invention will be described by way of example with reference to the accompanying drawings, in which:

FIG. 1 shows a perspective view of a system for determining one or more characteristics of a user based on an image of their eye, in particular their retina, and further in particular the Optic Nerve Head (ONH) region;

FIG. 2 shows a perspective view of a first embodiment of headset which forms part of the system for determining one or more characteristics of a user based on an image of their eye;

FIG. 3 shows a perspective view a second embodiment of headset which forms part of the system for determining one or more characteristics of a user based on an image of their eye;

FIG. 4 is a photograph showing an image of a user's eye, in particular their retina and showing an ONH and surrounding area;

FIG. 5 is a diagram illustrating the system for determining one or more characteristics of a user based on an image of their eye;

FIG. 6 is a flow diagram illustrating a method for determining one or more characteristics of a user based on an image of their eye;

FIG. 7a is a photograph showing a child's eye with the area around the ONH demonstrating features at the retina/vitreous gel interface reflecting the age of the child;

FIG. 7b is a photograph showing manual segmentation of the features referred to in FIG. 7(a), including the ONH, for training the algorithms for feature-specific age detection, including the outlined features;

FIGS. 8A to 8C shows a photograph with overlaid geometric patterns including a triangle, an ellipse and a square, to include the area surrounding the ONH within the 45 degrees, for depicting the cross section of features with the geometric patterns for training algorithms for disease change detection;

FIG. 9 is a photographic image of the optic nerve head of a patient with progressive glaucoma over ten years, demonstrating enlargement of the central pale area (cup) as the rim thins, with displacement of their blood vessels;

FIG. 10 illustrates OCT angiography (OCT-A) photographic images of a healthy optic nerve head vasculature (on the left) and on the right, a dark gap (between the white arrows) showing loss of vasculature of early glaucoma in a patient with no loss of visual fields;

FIG. 11a is an image of the optic nerve head divided into segments;

FIG. 11b illustrates a graph showing loss of neuroretinal rim according to age;

FIG. 12a is a process flow illustrating how an image of the optic nerve head is classified as healthy or at-risk of glaucoma by a dual neural network architecture, according to an embodiment of the present disclosure;

FIG. 12b is a process flow illustrating an image of the optic nerve head being cropped with feature extraction prior to classification, according to an embodiment of the present disclosure;

FIG. 13 is a flowchart illustrating an image classification process for biometric identification, according to an embodiment of the present disclosure;

FIG. 14a shows one circle of a set of concentric circles intersecting with the optic nerve head vasculature;

FIG. 14b is an image of concentric circles in a 200 pixel² segmented image intersecting with blood vessels and vector lines;

FIG. 15 is a concatenation of all blood vessel intersections for a given set of concentric circles—this is a feature set;

FIG. 16 illustrates an example of feature extraction with a circle at a radius of 80 pixels, according to an embodiment of the present disclosure;

FIG. 17 illustrates an example of a segmented image of optic nerve head vessels before and after a 4 degree twist with 100% recognition;

FIG. 18 illustrates a table of a sample feature set of resulting cut-off points in pixels at the intersection of the vessels with the concentric circles;

FIGS. 19a to 19c illustrate a summary of optic nerve head classification processes according to embodiments of the present disclosure;

FIG. 20 is a flowchart illustrating a computer-implemented method of classifying the optic nerve head, according to an embodiment of the present disclosure; and

FIG. 21 is a block diagram illustrating a configuration of a computing device which includes various hardware and software components that function to perform the imaging and classification processes according to the present disclosure.

### DETAILED DESCRIPTION

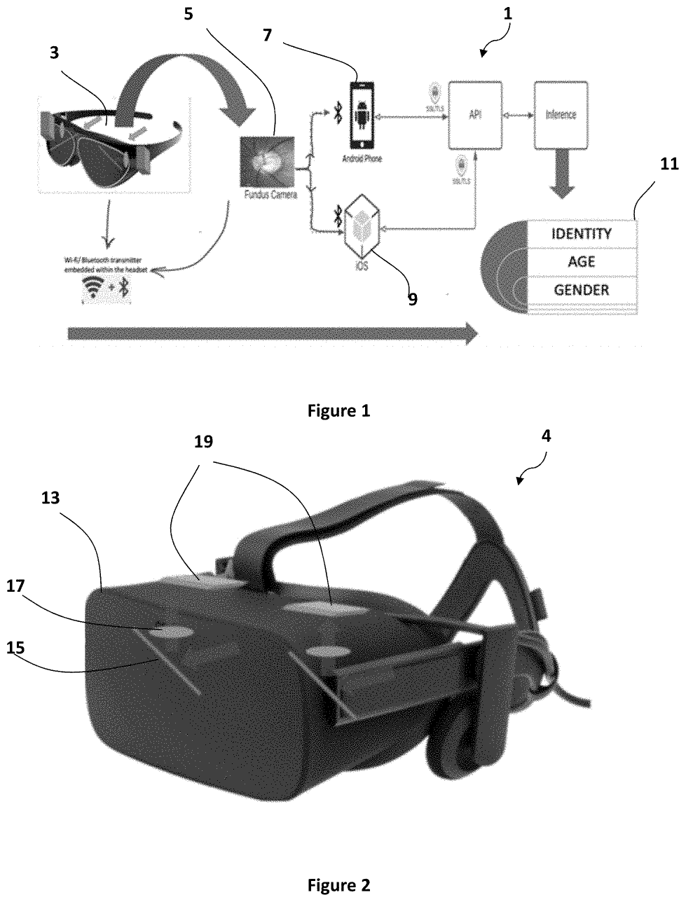

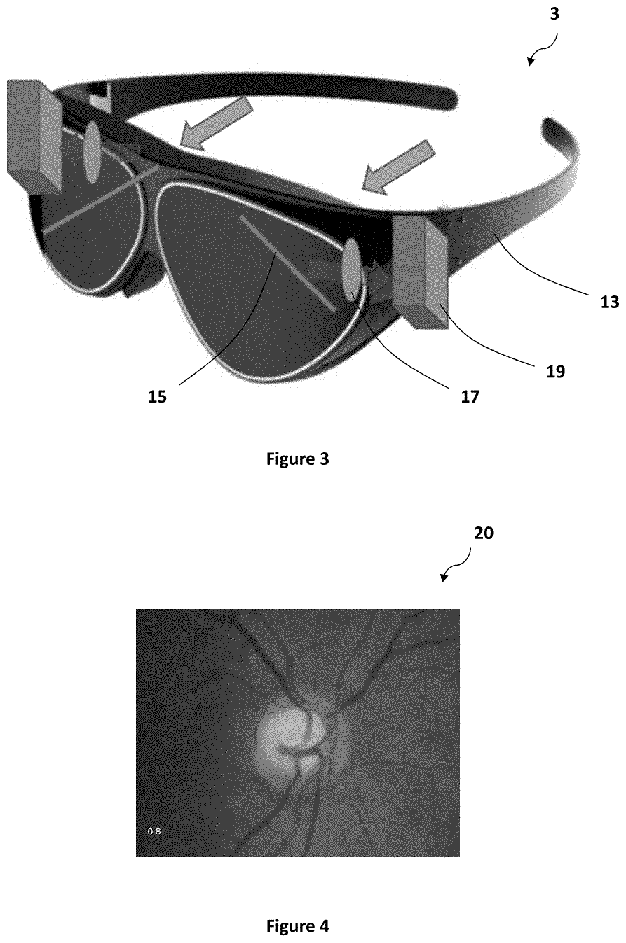

Referring now to the drawings, in particular FIG. 1, there is shown a system for determining one or more characteristics of a user based on an image of their eye which is generally indicated by the reference numeral 1. The system includes a headset 3, typically an Augmented Reality and/or Virtual reality (AR/VR) headset or any suitable head mounted set with camera for acquiring an image of the user's eye 5 when they are wearing the headset. Typically the AR/VR headset 3 may be a substantially helmet-like headset (such as that shown in FIG. 2 which is illustrated by the reference numeral 4) which encapsulates at least a portion of the user's head or alternatively the AR/VR headset 3 may be a pair of glasses (such as that shown in FIG. 3) providing AR/VR functionality. The AR/VR headset may be configured to provide both AR and VR functionality.

It should be understood that reference to a VR headset is intended to mean a headset which provides a virtual reality which can surround and immerse a person in a computer-generated, three-dimensional (3D) environment. The person enters this environment by wearing a VR headset which typically will include a screen and glasses or goggles that a user looks through when viewing a screen (e.g., a display device or monitor), gloves fitted with sensors, and external handheld devices that include sensors. Once the user enters the VR space, the person can interact with the 3D environment in a way (e.g., a physical way) that seems real to the person whether that's by use of external handheld devices or through the use of eye tracking or the like. Examples of VR headsets include those manufactured by Oculus® and Sony® but to name a few examples.

Further it should be understood that reference to an AR headset is intended to mean a headset which provides augmented reality (AR) which is an interactive experience of a real-world environment where the objects that reside in the real world are enhanced by computer-generated perceptual information, sometimes across multiple sensory modalities, including visual, auditory, haptic, somatosensory and olfactory. Examples of AR headsets include Microsoft Holo Lens® and Google Glass® but to name a few examples.

The headset 3, as well as including the components necessary for providing an AR or VR experience such as a screen, processing circuitry, speaker, memory, power supply etc., further includes an imaging device such as a camera 19 which is configured to acquire an image of the user's eye, in particular the user's retina, when the headset is worn by the user. An example of the image acquired by the camera is illustrated at reference numeral 5 within FIG. 1 as well as at FIGS. 4, 7 and 8 which show a photograph of a person's retina, in particular showing an Optic Nerve Head (ONH) and surrounding area. Typically the camera 19 is a Fundus camera, however it may alternatively be any camera suitable for acquiring an image of the user's retina, such as Ocular Computer Tomography (OCT), Ocular computer Tomography Angiography (OCT-A), LIDAR, near Infra Red imaging and any visual or sound wave imagery of the retina features. The camera 19 may be integrally coupled to the headset 3, however in an alternative embodiment, the camera 19 may be releasably coupleable to the headset 3 such as to allow for cameras 19 to be interchanged or updated as per the user's requirements. The headset 3 may further include one or more optical elements, such as beamsplitters, lenses such as objective or condenser lens, which are provided in conjunction with the camera 19 to aid in acquiring the image of the user's eye(s) whilst wearing the headset 3. For example as shown in FIGS. 2 and 3 the headsets 3, 4 typically include an optical assembly including at least at least one mirror 15 or other reflective element and a lens 17, the lens 17 typically being a convex lens, which define the image path between the camera 19 and the user's eyes.

The system 1 further includes a computing device 7, which is communicatively coupled to the camera 19, which is configured to: receive the image of the user's eye 5, in particular their retina; and determine one or more characteristics of the user based on the received image 5. The computing device 7 maybe be integrally connected to the headset 3, i.e. the computing device may be embedded or integrally attached to the headset 3 such that the determining of the one or more characteristics of the user based on the received image of the user's retina is performed entirely on the headset 3, which is then operable to display the determined characteristics to the user via the display of the headset itself. Additionally or alternatively, the computing device 7 may be located external/remote to the headset 3 and connectable via a wired connection such as to exchange data via wired transmission or further additionally or alternatively the headset 3 may include a wireless transmission means such as Wi-Fi®, Bluetooth®, other low power wireless transmission means or any other suitable wireless transmission means such that the headset 3 may wirelessly couple to the computing device 7 to exchange data, in particular image data of the user's retina such as shown at reference numerals 4 and 5 of FIG. 1. For example in this embodiment the computing device 7 may be in the form of a personal computing device such as a smartphone, tablet, laptop or any other suitable personal computing device which is wirelessly coupleable to the headset to exchange data such that the determining of the one or more characteristics of the user based on the received image of the user's retina is performed on the user's personal computing device, which is then operable to display the determined characteristics to the user via the display of their personal computing device. Further, additionally or alternatively the camera 19 may itself include wireless and/or wired transmission means for transmitting the data to the computing device or one or more further computing devices 7. The computing device 7 may also be configured to alert the user to or more characteristics determined based on the image of their eye. Additionally or alternatively the computing device 7 may be configured to monitor the user's determined eye characteristics over time.

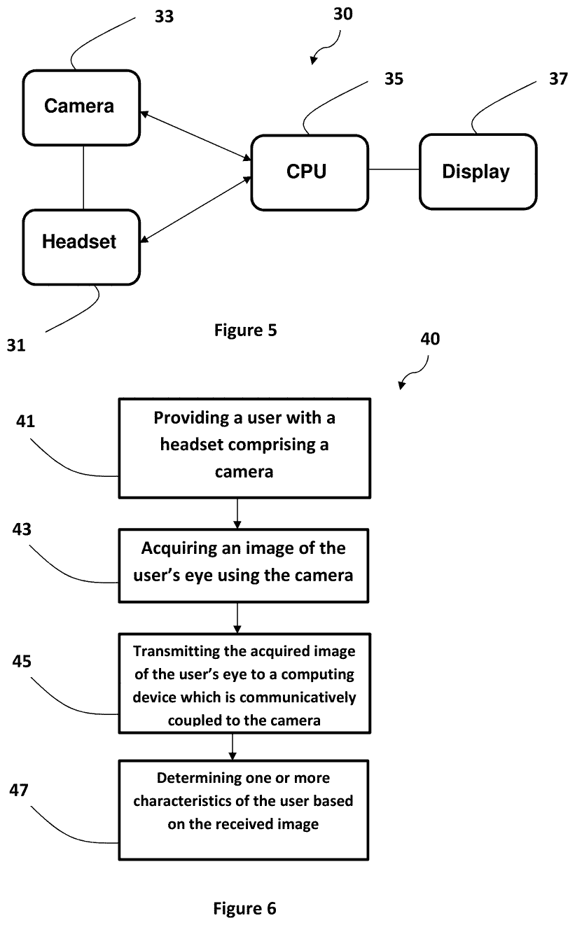

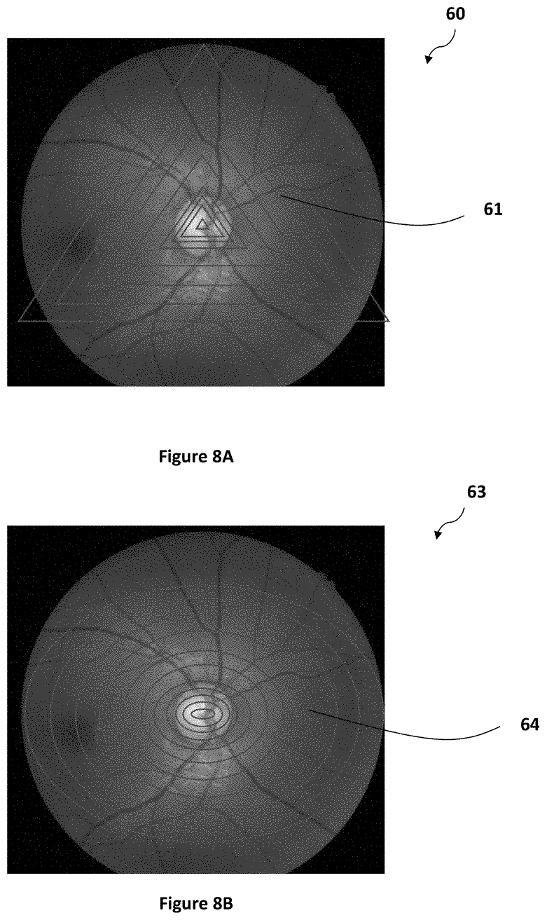

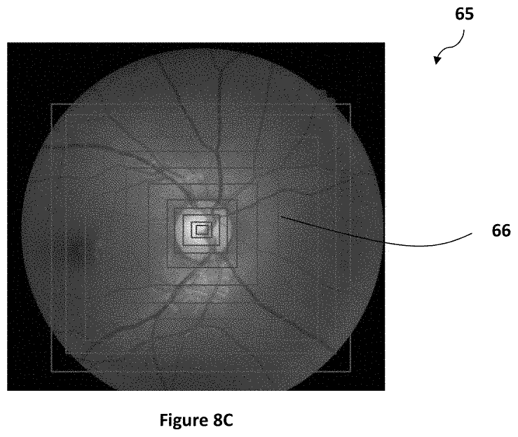

The computing device 7 is configured to receive the image of the user's eye and determine one or more characteristics of the user based on the received image. This is achieved by one or more deep neural networks or machine learning algorithms provided on or available to the computing device such as shown at reference numeral 11 of FIG. 1. Because of the well-known predictable changes which occur in the optic nerve head and surrounding area as a person ages, a well-trained deep learning model such as a convolutional neural network may handle image detection of such very effectively. The inherent commonality of image patterns of the retina, in particular the optic nerve head and surrounding area, across people of various ages and genders allows the deep neural network to effectively learn the characteristics associated with user's of different ages, genders etc. Hence, the deep neural network implemented by the computing device may be trained using training data, which includes a plurality of images of the optic nerve head from users of various ages, genders etc. The computing device 7 is typically configured to implement a computer-implemented method for classifying the optic nerve head which is suitable for determining the one or more characteristics of the user based on the received image, the computer-implemented method for classifying the optic nerve head being that as recited in the Applicant's other patents and patent applications including: EP3373798, U.S. Ser. No. 10/441,160, WO2018095994, IE S2016/0260 and US2018/0140180 each of which are herein incorporated by reference in their entirety. The Deep neural network may also be trained on the area including and surrounding the ONH up to 6 degrees field to incorporate the features of the retina/vitreous interface (as in FIG. 5a/5b) to specify the age of the adult or child and to differentiate between an adult and a child. The deep neural network may also be trained on the area surrounding the ONH up to 45 degrees field using the intersection of features with geometric shapes, as demonstrated in FIGS. 8A to 8C as is described in further detail herein.

The step of determining the one or more characteristics of the user based on the received image, which is typically implemented by the computing device 7 or any suitable processing means optionally including the method of classifying the optic nerve head 1000 as described above, the method including:
segmenting an image of an optic nerve head from a photographic image of an eye 1010;
segmenting the image of the optic nerve head into multiple segments each containing blood vessels and neuroretinal rim fibres 1020;
extracting features from the segmented images, the features describing relationships between the blood vessels themselves and between the blood vessels and the neuroretinal rim fibres in each of the segmented images 1030;
identifying characteristics of the optic nerve head based on the extracted features 1040; and
classifying the image of the optic nerve head based on the identified characteristics 1050.

Once the computing device has determined the one or more characteristics of the user, it may display these on the display of the VR/AR headset 3 or an external or remote display connected to a Head Mounted Set or the VR/AR headset via wired or wireless transmission means. Additionally or alternatively these may be provided audibly to the user through a speaker of the AR/VR headset or the computing device if this is separate therefrom.

The system 1 may be configured to acquire multiple different images of the user's eyes over a period of time and alert the user to changes occurring in the eye, in particular changes in characteristics of the eye, which may be indicative of eye disease or the like. For example, the computing device 7 may be configured, typically via pre-programming, to alert the user to acquire images of their eye's at predetermined periods of time e.g. once a week or once a year etc. Analysis of these images over the period of time allows for the detection of changes in characteristics of the eye images.

Alternatively the computing device 7 may be configured to alert the user to acquire images of their eyes at predetermined time intervals which are determined based on one or more previously determined characteristics of the user's eyes. For example, when the user's characteristics indicate that the user is an older person the computing device 7 may be configured to alert the user to acquire images of their eyes on a more regular basis. Additionally or alternatively, the headset 3, 4 may be configured to acquire the image of the user's eyes each time they wear the headset 3. It is envisioned that the headset 3 may be configured to perform other functions for the user as opposed to merely being used as an eye analysis and/or monitoring tool such that the operation of capturing the image of the user's eyes is as unobtrusive as possible and may be implemented discreetly as the user is wearing the headset for other purposes.

The one or more characteristics which are determined may include, one or more of: the age of the user, identity of the user, gender of the user, one or more health characteristics of the user such as the health of the user's eyes, the detection of one or more symptoms relating to one or more other health conditions i.e. diseases or injuries of the user not necessarily limited to the user's eye health. Further the one or more determined characteristics may be used as biometric identification information in third party software applications, typically as an identity and verification means. For example, determining one or more characteristics of the user based on the received image includes one or more of:
A) analysing the Optic nerve head pattern to identify the wearer of the glasses and/or to
B) classify the wearer as being of a specific age and/or being a child or an adult and/or being in a specific age band for example under 7 years/under 10 years/under 15 years etc.; and to
C) classify the likely gender of the wearer; and to
D) perform functions (A) to (C) glasses/headset/Augmented reality headset/virtual reality headset adapted for heads of animals to determine one or more characteristics of an animal;
E) and to make the classification results available within the glasses/headset/Augmented reality headset/virtual reality headset as projected within the Glasses viewing system for view by the wearer and/or
F) to make the classification results available within the glasses/headset/Augmented reality headset/virtual reality headset on a smart phone/computer screen via direct (plug in) connection cable or via remote transmitter, including Wi-Fi and or Bluetooth or any other remote image transmission system and
G) to include a voice/sound receiver and/or
H) voice/sound transmitter and/or remote connection to a
I) voice/sound transmitter/receiver with artificial intelligence analysis of voice/sound/automatic.

Referring again to FIG. 2 there is shown an AR/VR headset 4 in the form of a substantially helmet-like headset. This further illustrates the one or more Fundus cameras 19 which may be coupled thereon. The Fundus cameras 19 may be mounted at the top, bottom and/or either side of the head-mounted display of the headset. The Fundus images acquired by the cameras 19 may be projected with one or more optical elements onto the projection optics of the headset 3.

Similarly, FIG. 3 shows an embodiment of the AR/VR headset which is a more glasses shaped headset generally indicated by the reference numeral 3. A camera, typically a miniaturised camera 19, is mounted in the central part of the lenses or again with a reflective system similar to that shown to the left. One or more cameras for fundus imaging may be mounted at the top, bottom and/or either side of the head-mounted display of the headset. The Fundus images are projected by the optical elements onto the projection optics.

FIG. 4 is a photograph of an optic nerve head and part of the surrounding area (the image used may be plus or minus 45 degree field of view and macula or optic nerve head centered).

FIG. 5 is a diagram illustrating the system for determining one or more characteristics of a user based on an image of their eye generally indicated by the reference numeral 30. The system including a headset 31, camera 33 and a processor 35. The camera 33, as shown in FIGS. 1 to 3, is typically included within the headset 31 such that the camera 33 is configured to acquire an image of the user's eye when the user is wearing the headset 31. The processor 35 is typically a component of a computing device 7 such as that described in relation to FIG. 1. Optionally the system may also include a display such as a visual display unit or screen or the like which is communicatively coupled to the processor 35 which is configured to

Advantageously, the present invention provides glasses/headset/Augmented reality headset/virtual reality headset which will capture the image of the ONH and surrounding area, plus or minus 45 degrees, in order to use computer vision/artificial intelligence to determine one or more characteristics of the user such as to enroll/identify the wearer and provide automatic classification of the wearer as being a child or an adult and/or of a specific age/age band and/or male/female gender. The system can also be used as a global biometric for digital onboarding, for identity verification and for age band classification and child identification. Further advantageously, the system 1 of the present invention may be used as a health monitoring tool to monitor the characteristics of the user's eyes over a period of time and to alert the user to any changes in the characteristics.

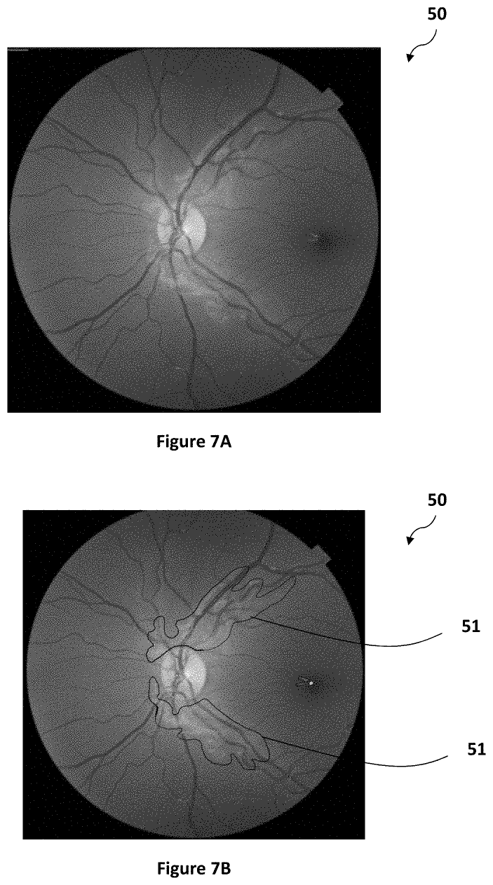

FIG. 7a is a photograph showing a child's eye with the area around the ONH demonstrating features at the retina/vitreous gel interface which reflect the age of the child. FIG. 7b is a photograph showing manual segmentation of the features referred to in FIG. 7a; this may be performed by a medical practitioner, including the ONH, for training the algorithms for feature-specific age detection, including the outlined features. FIGS. 8A to 8C show a photograph with overlaid geometric patterns including a triangle, an ellipse and a square, to include the area surrounding the ONH within the 45 degrees, for depicting the cross section of features with the geometric patterns for training algorithms for disease change detection. The geometric patterns segmenting the image of the eye for optimising the determination of characteristics therein. For example, referring to FIG. 7B in combination with FIGS. 8A to 8C the points at which the manually segmented features (see FIG. 7b) intersect with one or more of the concentric geometric patterns (as shown in FIGS. 8A to 8C) allows for the extraction of features and subsequent determination of one or more characteristics of the user's eye. Advantageously, the concentric geometric patterns which may be superimposed on the image of the user's eye are kept constant, such that they may be used as an accurate reference when assessing eye images from multiple people across multiple different age groups. The fixed nature of the concentric geometric patterns facilitating rapid and quick determination of features, the features including but not limited to blood vessels and branches thereof and intersection points between the blood vessels and branches thereof and the neuroretinal rim, within the images. This is particularly advantageous within the context of deep neural networks as it is provides an effective means of training the networks, as well as facilitating the implementation of trained neural networks in use. These aspects are described in further detail herein.

It will be understood that what has been described herein is an exemplary system for determining one or more characteristics of a user based on an image of their eye, in particular an image of the ONH and surrounding retina up to 45 degrees, using an AR/VR headset. While the present teaching has been described with reference to exemplary arrangements it will be understood that it is not intended to limit the teaching to such arrangements as modifications can be made without departing from the spirit and scope of the present teaching.

Further to the above the computer implemented method is for analysing, categorising and/or classifying relationships of characteristics of the optic nerve head axons and its blood vessels therein which are identified in the image of the user's eye which was acquired by the headset 3, in particular the camera(s) 17 coupled thereto, and based on this determining one or more characteristics of the user.

Machine learning and deep learning are ideally suited for training artificial intelligence to screen large populations for visually detectable diseases. Deep learning has recently achieved success on diagnosis of skin cancer and more relevant, on detection of diabetic retinopathy in large populations using 2D fundus photographs of the retina. Several studies have previously used machine learning to process spectral-domain optical coherence tomography (SD-OCT) images of the retina. Some studies have used machine learning to analyse 2D images of the optic nerve head for glaucoma, including reports of some success with deep learning. Other indicators of glaucoma which have been analysed with machine learning include visual fields, detection of disc haemorrhages and OCT angiography of vasculature of the optic nerve head rim.

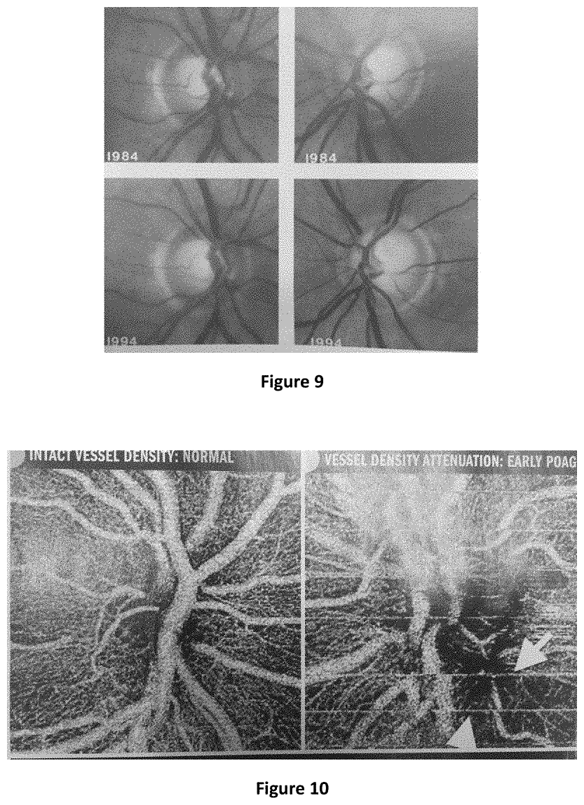

The computer-implemented method for classifying the optic nerve head uses convoluted neural networks and machine learning to map the vectors between the vessels and their branches and between the vessels and the neuroretinal rim. These vectors are constant and unique for each optic nerve head and unique for an individual depending on their age. FIGS. 9 and 10 demonstrate results of change in the neuroretinal rim with age by analysing change in each segment of the rim. As the optic nerve head grows, the position of the blood vessels and their angles to each other changes, and thus their relationship vectors will change as the relationships to the blood vessels and to the axons change. The artificial intelligence is also trained with an algorithm to detect changes in the relationship of the vectors to each other, and to the neuroretinal rim, so that with that loss of axons, such as with glaucoma, change will be detected as a change in the vectors and an indicator of disease progression.

The computer-implemented method may include computer vision algorithms, using methods such as filtering, thresholding, edge detection, clustering, circle detection, template matching, transformation, functional analysis, morphology, etc., and machine learning (classification/regression, including neural networks and deep learning) to extract features from the images and classify or analyse the features for the purposes described herein.

The algorithms may be configured to clearly identify the optic disc/nerve head as being most likely to belong to a specific individual to the highest degree of certainty as a means of identification of the specific individual for the purposes of access control, identification, authentication, forensics, cryptography, security or anti-theft. The method may use features or characteristics extracted from optic disc/nerve images for cryptographic purposes, including the generation of encryption keys. This includes the use of a combination of both optic discs/nerves of an individual.

The algorithms may be used to extract features or characteristics from the optic disc/nerve image for the purposes of determining the age of a human or animal with the highest degree of certainty for the purposes of security, forensics, law enforcement, human-computer interaction or identity certification.

The algorithms may be designed to analyse changes in the appearance of the optic nerve disc head/volume attributable to distortion due to inherent refractive errors in the eyeball under analysis. The algorithm may be configured to cross reference inherent changes in size, for example, bigger disc diameter than normal database, smaller disc diameters than normal database, tilted disc head.

The algorithms may include calculation and analyses of ratio of different diameters/volume slices at different multiple testing points to each other within the same optic nerve head, and observing the results in relation to inherent astigmatism and refractive changes within the eyeball of the specific optic nerve. Refractive changes can be due to shape of the eyeball, curvature and power of the intraocular lens and/or curve and power of the cornea of the examined eyeball.

The algorithm may include the detection of a change of artery/vein dimensions as compared with former images of the same optic nerve head vessels and/or reference images of healthy optic nerve head blood vessels.

The algorithm may be used for the purposes of diagnosing changes in artery or vein width to reflect changes in blood pressure in the vessels and/or hardening of the vessels.

The algorithms may be applied to the optic nerve head of humans, of animals including cows, horses, dogs, cats, sheep, and goats; including uses in agriculture and zoology.

The algorithms may be used to implement a complete software system used for the diagnosis and/or management of glaucoma or for the storage of and encrypted access to private medical records or related files in medical facilities, or for public, private or personal use.

The algorithms may be configured to correlate with changes in visual evoked potential (VEP) and visual evoked response (VER) as elicited by stimulation of the optic nerve head before, after or during imaging of the optic nerve head.

The algorithms may also model changes in the response of the retinal receptors to elicit a visual field response/pattern of the fibres of the optic nerve head within a 10 degree radius of the macula including the disc head space.

The algorithms may be adapted to analyse the following:
1. Appearance/surface area/pattern/volume of the average optic disc/nerve head/vasculature for different population groups and subsets/racial groups, including each group subset with different size and shaped eyes, including myopic/hypermetropic/astigmatic/tilted disc sub groups, different pigment distributions, different artery/vein and branch distributions, metabolic products/exudates/congenital changes (such as disc drusen/coloboma/diabetic and hypertensive exudates/haemorrhages.
2. Differences in appearance/surface area/pattern/volume of the optic disc/nerve head/vasculature when compared to the average in the population.
3. Differences in appearance/surface area/pattern/volume of the optic disc/nerve head/vasculature when compared to previous images/information from the same patient in the population.
4. Appearance/surface area/pattern/volume of the optic nerve head/vasculature anterior and including the cribriform plate for different population groups and subsets/racial groups, including each group subset with different size and shaped eyes, including myopic/hypermetropic/astigmatic/tilted disc sub groups, including different pigment distributionism, including different artery/vein and branch distributions, including metabolic products/exudates/congenital changes (such as disc drusen/coloboma/diabetic and hypertensive exudates/haemorrhages.
5. Differences in appearance/surface area/pattern/volume of the optic nerve head/vasculature anterior and including the cribriform plate for different population groups and subsets/racial groups, including each group subset with different size and shaped eyes, including myopic/hypermetropic/astigmatic/tilted disc sub groups, including different pigment distributions, including different artery/vein and branch distributions, including metabolic products/exudates/congenital changes (such as disc drusen/coloboma/diabetic and hypertensive exudates/haemorrhages when compared to the average in the population.
6. Differences in appearance/surface area/pattern/volume of the optic nerve head/vasculature anterior and including the cribriform plate for every different population groups and subsets/racial groups, including each group subset with different size and shaped eyes, including myopic/hypermetropic/astigmatic/tilted disc sub groups, including different pigment distributions, including different artery/vein and branch distributions, including metabolic products/exudates/congenital changes (such as disc drusen/coloboma/diabetic and hypertensive exudates/haemorrhages when compared to previous images/information from the same patient in the population.
7. Classifying the remaining optic nerve head and associated vasculature and the ten millimetres deep to the surface, as being normal/abnormal; as being at a high probability of representing a damaged nerve head, as being a volume which is abnormal in relation to the position of other factors at the posterior pole of the fundus, factors/patterns such as distance of the optic nerve head and/or vasculature and rim to the macula; distance to the nasal arcade of arteries and veins, distance to the temporal arcade of veins and arteries.
8. Describing the patterns representing the likelihood of the relationship of the optic nerve outer rim/inner rim/cup/rim pigment/peripapillary atrophy to the fundus vessels/macula as being abnormal; as having changed when compared to an image of the same fundus taken at an earlier time or later time.
9. Attributing the likelihood of the measured volume of optic disc/nerve/vasculature visible to the examiner's eye/camera lens or as measured by OCT/OCT-Angiography as being diagnostic of glaucoma/at risk for glaucoma (all sub groups of glaucoma) and all group of progressive optic nerve disorders/degenerative optic nerve disorders including neuritis/disseminated sclerosis/; as being evidence of being a lower or higher nerve head volume when compared to earlier or later volume or surface area measurements of the same optic nerve head, or being compared to a database/databases of normal, diseased or damaged optic nerve head, in every population subset and racial distribution, particularly Caucasian, Asian, south Pacific and all African races/descendants.
10. Attributing the likelihood of the measured volume/area of optic disc/nerve/vasculature visible to the examiner's eye/camera lens or as measured by OCT/computer vision technology, as being evidence of being a lower or higher nerve head volume when compared to earlier or later volume or surface area measurements of the same optic nerve head, or being compared to a database/databases of normal, diseased or damaged optic nerve head, in every population subset and racial distribution, particularly Caucasian, Asian, south Pacific and all African races/descendants, for all age related changes to the optic nerve/central nervous system, in particular, Alzheimer's disease and diabetic neuropathy and infective nerve disorders such as syphilis/malaria/zika viruses.
11. Clearly identify the optic disc/nerve head and vasculature as being most likely to belong to a specific individual to the highest degree of certainty.
12. Clearly identify the optic disc/nerve head and vasculature as being most likely to belong to a specific individual to the highest degree of certainty as a means of identification of the specific individual for secure access to any location, virtual or special/geographic. For example,
a) to replace fingerprint access to electronic/technology innovations, as in mobile phones/computers; to replace password/fingerprint/face photography for secure identification of individuals accessing banking records/financial online data/services.
b) to replace fingerprint access to electronic/technology innovations, as in mobile phones/computers; to replace password/fingerprint/face photography for secure identification of individuals accessing Interpol/international/national security systems
c) to replace fingerprint access to electronic/technology innovations, as in mobile phones/computers; to replace password/fingerprint/face photography for secure identification of individuals accessing health records/information data storage/analysis.

As mentioned previously, to determine the one or more characteristics of the user's eye obtained from the camera the present disclosure uses a computer-implemented method of classifying the image of the user's eye, in particular the optic nerve head and typically also the surrounding area thereof, the method including operating one or more processors to: segment an image of an optic nerve head from a photographic image of an eye; segment the image of the optic nerve head into multiple segments each containing blood vessels and neuroretinal rim fibres; extract features from the segmented images, the features describing relationships between the blood vessels themselves and between the blood vessels and the neuroretinal rim fibres in each of the segmented images; identify characteristics of the optic nerve head based on the extracted features; and classify the image of the optic nerve head based on the identified characteristics. Optionally, to determine the one or more characteristics of the user based on the received image the computing device is configured to: segment the image of the user's eye into multiple segments, superimpose multiple concentric geometric patterns onto the multiple segments; extract features from the segmented images, the features including elements of the eye which intersect with one or more concentric geometric patterns; and identify characteristics of the eye based on the extracted features.

It will be understood in the context of the present disclosure that for the purposes of classifying the optic nerve head, the optic nerve head includes the optic nerve head (optic disc) itself and the associated vasculature including blood vessels emanating from the optic nerve head. The optic nerve head also includes neuroretinal rim fibres located in the neuroretinal rim. It will also be understood that image segmentation is the process of dividing or partitioning a digital image into multiple segments each containing sets of pixels. The goal of segmentation is to simplify and/or change the representation of an image into something that is more meaningful and easier to analyse.

The method involves identification of the region of interest, that is the optic nerve head and its vasculature. A deep neural network may be used to segment the image of the optic nerve head and associated blood vessels. The method uses a Deep Neural Network for segmentation of the image. As a non-limiting example, Tensorflow® from Google Python® library was used as follows. Results on a small sample training set had a Sorensen-Dice coefficient of 75-80%.

The method includes automatic high-level feature extraction and classification of the image, for any of the purposes described herein (identification, age determination, diagnosis of optic nerve head vessels and/or axonal fibre loss and/or changes) or a second deep neural network trained to use artificial intelligence to identify/classify the image, for any of the purposes described herein (identification, age determination, diagnosis of optic nerve head vessels and/or axonal fibre loss and/or changes).

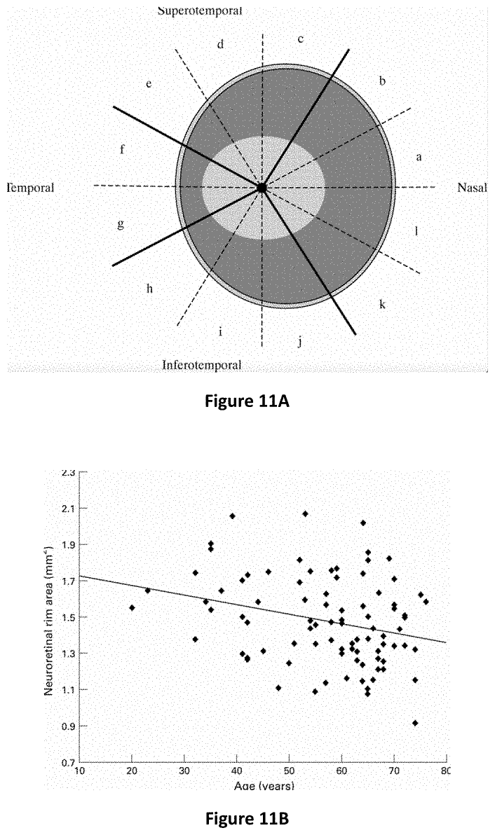

Once the image of the optic nerve head and its vasculature is segmented from the image of the eye, the optic nerve head image is further segmented according to the blood vessels within and the optic nerve head neuroretinal rim fibres. Segmentation of the optic nerve head image is illustrated in FIG. 11a. Features are extracted from the segmented images, the features including relationships between the vessels themselves and between the blood vessels and the neuroretinal rim. The segmenting the image of the optic nerve head into multiple segments includes using at least one of machine learning, deep neural networks, and a trained algorithm to automatically identify at least one of i) blood vessel patterns and ii) optic nerve head neuroretinal rim patterns. The relationships between the vessels themselves and between the blood vessels and the neuroretinal rim are described using vectors mapped between points on the blood vessels and the neuroretinal rim in each of the segmented images.

At least one of machine learning, deep neural networks and a trained algorithm may be used to automatically identify the image of at least one of the i) blood vessel patterns and ii) optic nerve head neuroretinal rim patterns as specifically belonging to an individual eye image at that moment in time. The optic nerve head image may be classified as being likely to be glaucomatous or healthy. The optic nerve head image may be classified as being likely to belong to an adult or a child. It may be identified when the image changes i.e. develops changes to blood vessel relationship and/or optic nerve fibre head, or has changed from an earlier image of the same optic nerve head, such as with disease progression and/or ageing.

The method of the present disclosure can map the vessel relationships and predict the most likely age category of the optic nerve head being examined based on the set of ratios of vessels and vessel to rim and the algorithms form the deep learning data base processing. The neuroretinal rim thickness decreases with age while the position of the vessels will and vector rim distances will drift. FIG. 11b illustrates a graph showing loss of neuroretinal rim according to age. Children's optic nerve heads have a different set of vector values compared to adults.

In more detail, the method may include, for each segment: superimposing multiple concentric geometric patterns, the geometric patterns including but not limited to circles, ellipses, squares, or triangles, on the segment such as shown for example in FIGS. 8A to 8C; determining intersection points of the geometric patterns such as the circles with blood vessels and branches thereof and intersection points between the blood vessels and branches thereof and the neuroretinal rim; mapping vectors between the intersection points; determining distances of the vectors; determining ratios of the vector distances; combining sequences/permutations of the ratios into an image representation; searching a lookup table for the closest representation to the image representation; and classifying the optic nerve head according to the closest representation found.

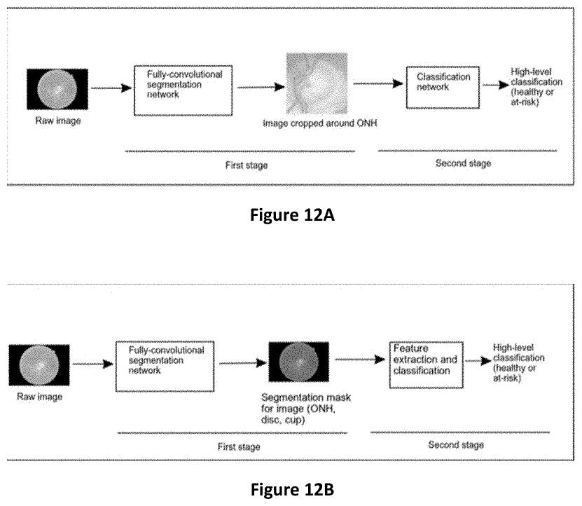

Several embodiments of the system are detailed as follows. In one embodiment, as illustrated in FIG. 12a, the image is classified as healthy or at-risk of glaucoma by dual neural network architecture.
1. A 2D photographic image of an eye may be obtained using a 45 degree fundus camera, a general fundus camera, an assimilated video image, or a simple smartphone camera attachment, or a printed processed or screen image of the optic nerve head, or an image or a photograph of an OCT-A image of an optic nerve head, from either a non-dilated or dilated eye of a human or any other eye bearing species with an optic nerve. A first fully convolutional network may locate the optic nerve head by classifying each pixel in the image of the eye.
2. The fully convolutional network then renders a small geometric shape (e.g. circle) around the optic nerve head and crops the image accordingly.
3. This resulting image can be fed to a trained second convolutional neural network, or have manual feature extraction, which makes a high-level classification of the optic nerve head as healthy or at risk of glaucoma.

In a further embodiment as illustrated in FIG. 12b:
1. A first fully convolutional network identifies a fixed area around the vessel branch patterns.
2. The image is then cropped accordingly and a variety of features are extracted from the resulting image including the vessel to vessel and vessel to nerve fibre ratios.
3. The image is classified as adult or child, and/or including the ability to detect changes with age on the same image in subsequent tests and therefore identify the age of the optic nerve head being segmented using artificial intelligence and/or manual feature extraction.

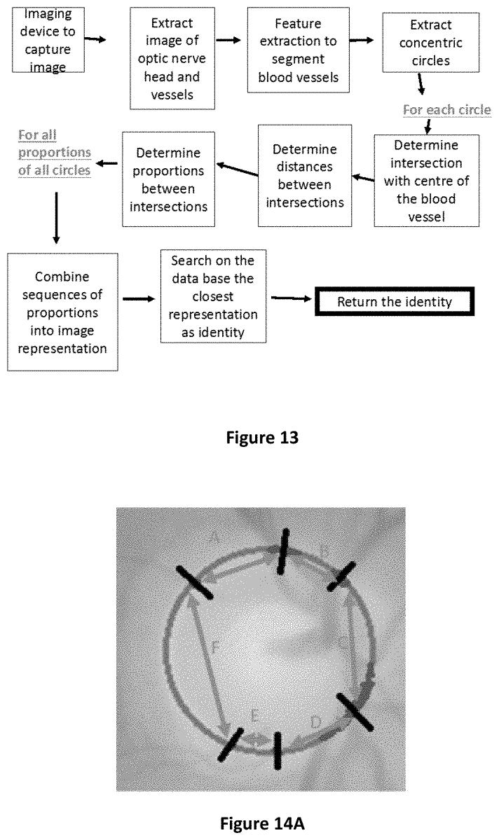

FIG. 13 is a flowchart illustrating an image classification process for biometric identification, according to an embodiment of the present disclosure. Referring to FIG. 13, the image classification process according to the present embodiment includes using an imaging device to capture an image of the eye 110, segmenting an image of the optic nerve head and its vasculature from the eye image 120, using feature extraction to segment the blood vessels 130, superimposing concentric geometric patterns, in this case circles, on each of the segmented images 140, for each circle, determining intersection points of the circle with the blood vessels and neuroretinal rim 150, determining distances between the intersection points 160, determining proportions of the distances 170, combining sequences/permutations of the proportions into an image representation 180, and searching a database or lookup table for the closest representation as an identity of the optic nerve head 190 and returning the identify of the optic nerve head 200.

As an experimental non-limiting working example of image classification, the methodology of the present disclosure is further described by reference to the following description and the corresponding results. A data set consisted of 93 optic nerve head images taken at 45 degrees with a fundus camera (Topcon Medical Corporation) with uniform lighting conditions. Images were labelled by ophthalmologists as being healthy or glaucomatous based on neuroretinal rim assessment. Criteria for labelling were based on RetinaScreen. Glaucoma was defined as a disc >0.8 mm in diameter and/or difference in cup-disc ratio of 0.3, followed by ophthalmologist examination and confirmation. The technique was first proofed for 92% concordance with full clinical diagnosis of glaucoma being visual field loss and/or raised intraocular pressure measurements.

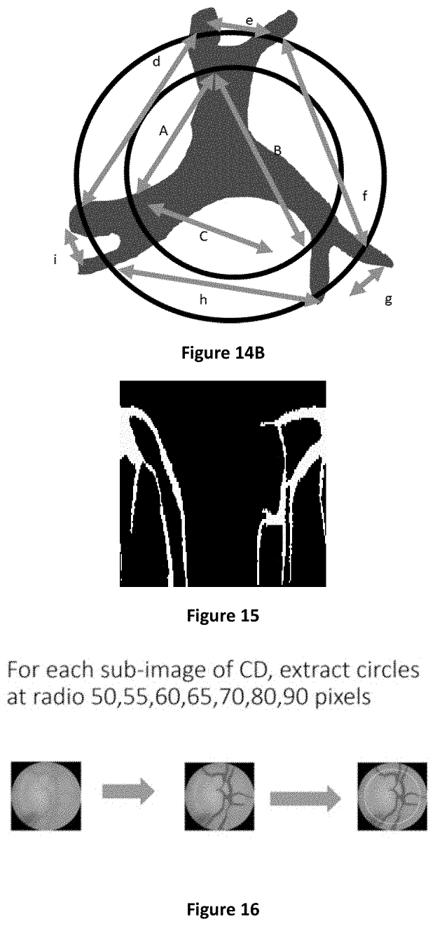

The first step, pre-processing, involves a fully convolutional network cropping the image of the eye to a fixed size around the optic nerve head at the outer neuroretinal rim (Elschnig's circle). The blood vessels are manually segmented (see FIG. 11a) into individual blood vessels and branches thereof. Multiple concentric circles are superimposed on each of the segmented images and the intersection of a circle with a specific point on the centre of a blood vessel is extracted, as illustrated in FIGS. 14a and FIG. 14b. FIG. 14a shows one circle of a set of concentric circles intersecting with the optic nerve head vasculature. Note the angle between the axes and the vectors reflects changes in direction of the vessel position, as with change in neuroretinal rim volume which causes vessels to shift. FIG. 14b is an image of concentric circles in a 200 pixel² segmented image intersecting with blood vessels and vector lines.

FIG. 15 is a concatenation of all blood vessel intersections for a given set of concentric circles—this is the feature set. This image is used to match against other feature set images in a database. The Levenstein distance is used to do the similarity match. The image with the lowest Levenstein distance is deemed to be the closest match. A sample feature set is shown in FIG. 16 and the table in FIG. 18. A summary of intersection points is generated from the extracted concentric circles from the center of the optic nerve head in the image of FIG. 12. The white area represents the blood vessels. For each circle 100 points may be extracted, which correspond to an area that belongs to a blood vessel (white), and black relates to intervascular space along the circles. The top border of the picture corresponds to the circle of radius=1 pixel; the lower border corresponds to the circle of radius=100 pixels. FIG. 18 illustrates a table of a sample feature set of resulting cut-off points in pixels at the intersection of the vessels with the concentric circles.

In one example, seven concentric circles may be superimposed on the segmented image from the centre of the optic nerve head with respective ratios of 50, 55, 60, 65, 70, 80 and 90 pixels. The intersection of the circles with the blood vessels is mapped, as illustrated in the flow diagram of FIG. 13, and summarised as shown in FIG. 14. The proportions are calculated using machine learning to classify the extracted sequences and/or permutations of proportions to 1-nearest neighbour (k-NN). k-NN also known as K-Nearest Neighbours is a machine learning algorithm that can be used for clustering, regression and classification. It is based on an area known as similarity learning. This type of learning maps objects into high dimensional feature spaces. The similarity is assessed by determining similarity in these feature spaces (we use the Levenstein distance. The Levenstein distance is typically used to measure the similarity between two strings (e.g. gene sequences comparing AATC to AGTC would have a Levenstein distance of 1). It is called the edit distance because it refers to the number of edits that are required to turn one string into another.

The sequences/permutations of proportions is used as the sequence of original features for the optic disc image.
Example of vector of distances=[A, B, C, D, E, F]
Example of vector of proportions [A/B, B/C, C/D, E/F, F/A].

For each picture, the set of nine vectors of proportions represents its feature set. FIGS. 9 and 11. Adversarialism was challenged with a 4 degree twist as illustrated in FIG. 13. Adversarialism is the result of a small visually undetectable change in pixels in the image being examined, which in 50% of cases causes convoluted neuronal network algorithms to classify the image as a different one (e.g. a missed diagnosis in a diseased eye). Despite the twist to alter the pixels, the result was still 100% accurate because the change maintained the correct vector relationships which establish the unique identity of the optic nerve fibre head and therefore the reliability of the invention. Levenstein distance is used to compare the sequences of proportions, where the atomic cost of swapping two proportions is the square value of the difference of the logarithms of the proportions:

Atomic cost=(log(a)−log(b))\hat{ }2 (the cost of swapping two proportions of different value)
Each insertion of deletion has a cost of one unit.

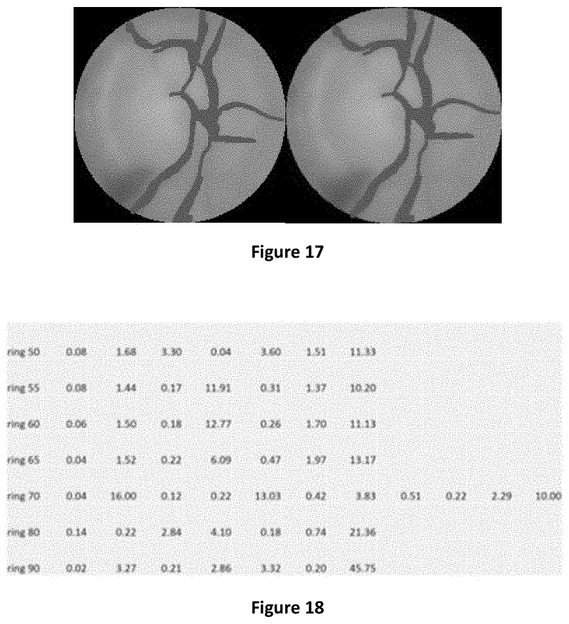

The results are illustrated in FIG. 17. The k-NN algorithm was trained with all 93 pictures. The algorithm was then used to identify an image from the set as being the particular labelled image. 100% of images selected were accurately identified. The images from the training set were then twisted 4 degrees, to introduce images separate to the training set. The algorithm was then challenged to correctly identify the twisted images and accuracy per labelled image was 100%. Taking the correct and incorrect classification as a binomial distribution and using the Clopper-Pearson exact method, it was calculated that with 95% confidence the accuracy of the system is between 96% and 100%.

The Clopper-Pearson exact method uses the following formula:

$${( 1 + \frac{n - x + 1}{xF ( 1 - α / 2 ; 2 x , 2 ( n - x + 1 )} )}^{- 1} < p < {( 1 + \frac{n - x}{( x + 1 ) F ( α / 2 ; 2 ( x - 1 ) , 2 ( n - x ) )} )}^{- 1}$$

where x is the number of successes, n is the number of trials, and F(c; d1, d2) is the 1−c quantile from an F-distribution with d1 and d2 degrees of freedom.

Note, the first part of the equation is the lower range for the interval and the second then highest, which in this case is 100%.

Traditional machine learning and deep learning in the region of the optic nerve head and the surrounding retina has not identified the relationships within the optic nerve head of the vessels and axons to each other, nor has any used the relationships for biometric identification or optic disc age assessment. Some studies have been performed with three dimensional frequency domain optical coherence tomography (FD-OCT) imaging, which only has achieved 62% sensitivity in screening tests for glaucoma and 92% in clinical sets. Others, such as the present disclosure, use 2D fundus photographs of the retina and optic nerve head. The present disclosure provides the ability to uniquely identify the optic nerve head and its vasculature in order to be able to screen for changes to the optic nerve head and blood vessels with a minimum of 95% specificity and a sensitivity greater than 85% to avoid missing a blinding preventable condition such as glaucoma. Almost all work with traditional machine learning and recent deep learning makes a diagnosis of glaucoma based on a small clinical set commenting only on the vertical cup disc ratio and in a few, textural analysis. Data sets have excluded the general population with all the ensuing morphological and refractive variations, precluding any sensitivity for screening the general population. As mentioned, none has the power to 100% identify the optic nerve head, as with the present disclosure. Identification means the power to state ‘not the same’ as previous disc identification, i.e., to say the optic nerve head has changed. Almost all studies prior to the present disclosure have analysed the optic nerve head for glaucoma disease and not basic optic nerve head vessels to neuroretinal rim relationship. Furthermore, they have focused on what is called the cup-disc ratio using segmentation of the disc outer rim minus the inner cup, as a glaucoma index. However, a cup-disc ratio is not definitively due to axonal optic nerve fibre loss and furthermore, the ratio is a summary of the measurement of a specific radius of a disc which is rarely a perfect circle. It is also well accepted amongst ophthalmologists that although an increased optic cup-disc ratio suggests a risk of glaucoma, there is a high chance of over fitting with a labelled data set from patients already diagnosed, with an unacceptable chance that glaucoma can progress with loss of axons without affecting the cup/disc ratio.

There are a number of possible applications of the methods described herein as follows. One application is to clearly identify the optic nerve head and its vasculature as being most likely to belong to a specific individual to the highest degree of certainty. Here, the second stage of the method is a convolutional neural network trained on a large dataset of fundus images (cropped by a fully convolutional network at the first stage to a fixed geometric shape around the optic nerve head or, in an alternative configuration, cropped to a fixed area around the optic nerve head vessel branch patterns) labeled with identities (with multiple images for each identity) to produce a feature vector describing high-level features on which optic nerve heads can be compared for similarity in order to determine identity. The method may use features or characteristics extracted from optic nerve head images for cryptographic purposes, including the generation of encryption keys. This includes the use of a combination of both optic discs/nerves/vessels of an individual, or as a means of identification of the specific individual for the purposes of use as a biometric, use online to allow access to secure online databases, use with any device to access the device, use with any device to access another device (for example a car). This may be done as a means of identification of the specific individual for secure access to any location, either in cyberspace or through a local hardware device receiving the image of the individual's optic nerve head directly. For example, to replace or be used in combination with other biometric devices, such as fingerprint/retina scan/iris scan in order to access electronic devices such as mobile phones or computers.

Another application can be to determine the age of a human or animal with the highest degree of certainty for the purposes of security, forensics, law enforcement, human-computer interaction or identity certification. Here, the second stage of the method is a convolutional neural network trained on a large dataset of fundus images (cropped by a fully convolutional network at the first stage to a fixed geometric shape around the optic nerve head or, in an alternative configuration, cropped to a fixed area around the optic nerve head vessel branch patterns) labelled for age which can take a new fundus image and classify the age of the individual.

In addition to humans, the algorithms may be applied to the optic nerve head of animals/species including cows, horses, dogs, cats, sheep, and goats; including uses in agriculture and zoology. The algorithms may be used to implement a complete software system used for the diagnosis and/or management of glaucoma or for the storage of and encrypted access to private medical records or related files in medical facilities, or for public, private or personal use.

The methodology of the present disclosure may be used to detect changes as the neuroretinal rim area reduces with age. This will have an important role in cybersecurity and the prevention of cyber-crimes relating to impersonation and/or inappropriate access to the internet to/by children.

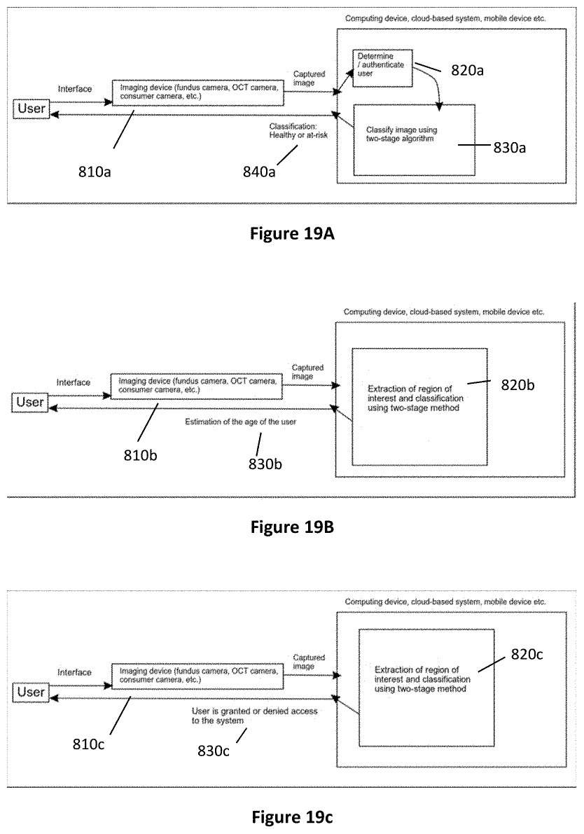

FIGS. 19a to 19c illustrate a summary of optic nerve head classification processes according to embodiments of the present disclosure. Referring to FIG. 19a, a first process includes capturing an image of the optic nerve head using an imaging device 810a, determining or authenticating the user 820a, classifying the optic nerve head using a two-stage algorithm as described above 830a, and classifying the optic nerve head as healthy or at-risk 840a. Referring to FIG. 19b, a second process includes capturing an image of the optic nerve head of a user using an imaging device 810b, extracting a region of interest using a two-stage algorithm as described above 820b and, and estimating the age of the user 830b. Referring to FIG. 19c, a third process includes capturing an image of the optic nerve head of a user using an imaging device 810c, extracting a region of interest using a two-stage algorithm as described above 820c and, and granting or denying the user access to a system 830c.

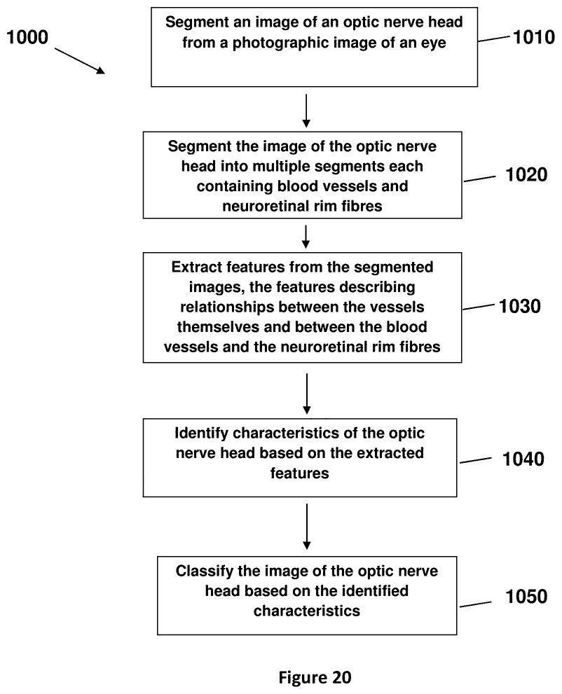

FIG. 20 is a flowchart illustrating a computer-implemented method 1000 of classifying the optic nerve head which is used to determine the one or more characteristics of the user based on the image of their eye. Referring to FIG. 20, the method includes operating one or more processors to: segment an image of an optic nerve head from a photographic image of an eye 1010; segment the image of the optic nerve head into multiple segments each containing blood vessels and neuroretinal rim fibres 1020; extract features from the segmented images, the features describing relationships between the blood vessels themselves and between the blood vessels and the neuroretinal rim fibres in each of the segmented images 1030; identify characteristics of the optic nerve head based on the extracted features 1040; and classify the image of the optic nerve head based on the identified characteristics 1050.

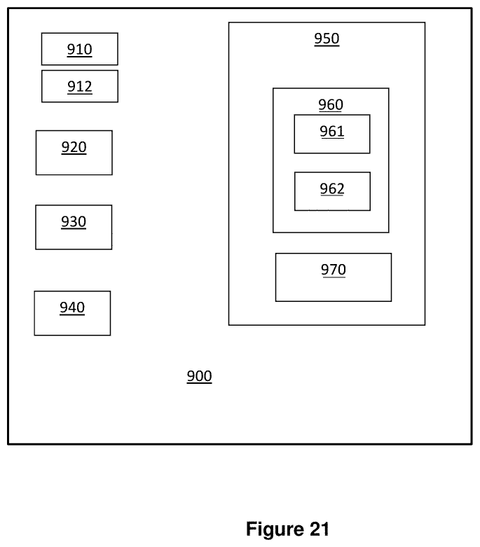

FIG. 21 is a block diagram illustrating a configuration of a computing device 900 which includes various hardware and software components that function to perform the imaging and classification processes according to the present disclosure. The computing device 200 may be a personal computing device such as a smartphone, laptop, tablet or the like or the computing device 200 may be integrated within the headsets 3, 4 shown at FIGS. 2 and 3 of the drawings. Referring to FIG. 20, the computing device 900 includes a user interface 910, a processor 920 in communication with a memory 950, and a communication interface 930. The processor 920 functions to execute software instructions that can be loaded and stored in the memory 950. The processor 920 may include a number of processors, a multi-processor core, or some other type of processor, depending on the particular implementation. The memory 950 may be accessible by the processor 920, thereby enabling the processor 920 to receive and execute instructions stored on the memory 950. The memory 950 may be, for example, a random access memory (RAM) or any other suitable volatile or non-volatile computer readable storage medium. In addition, the memory 950 may be fixed or removable and may contain one or more components or devices such as a hard drive, a flash memory, a rewritable optical disk, a rewritable magnetic tape, or some combination of the above.

One or more software modules 960 may be encoded in the memory 950. The software modules 960 may include one or more software programs or applications having computer program code or a set of instructions configured to be executed by the processor 920. Such computer program code or instructions for carrying out operations for aspects of the systems and methods disclosed herein may be written in any combination of one or more programming languages.

The software modules 960 may include at least a first application 961 and a second application 962 configured to be executed by the processor 920. During execution of the software modules 960, the processor 920 configures the computing device 900 to perform various operations relating to the embodiments of the present disclosure, as has been described above.

Other information and/or data relevant to the operation of the present systems and methods, such as a database 970, may also be stored on the memory 950. The database 970 may contain and/or maintain various data items and elements that are utilized throughout the various operations of the system described above. It should be noted that although the database 970 is depicted as being configured locally to the computing device 900, in certain implementations the database 970 and/or various other data elements stored therein may be located remotely. Such elements may be located on a remote device or server—not shown, and connected to the computing device 900 through a network in a manner known to those skilled in the art, in order to be loaded into a processor and executed.

Further, the program code of the software modules 960 and one or more computer readable storage devices (such as the memory 950) form a computer program product that may be manufactured and/or distributed in accordance with the present disclosure, as is known to those of skill in the art.

The communication interface 940 is also operatively connected to the processor 920 and may be any interface that enables communication between the computing device 900 and other devices, machines and/or elements. The communication interface 940 is configured for transmitting and/or receiving data. For example, the communication interface 940 may include but is not limited to a Bluetooth, or cellular transceiver, a satellite communication transmitter/receiver, an optical port and/or any other such, interfaces for wirelessly connecting the computing device 900 to the other devices.

The user interface 910 is also operatively connected to the processor 920. The user interface may include one or more input device(s) such as switch(es), button(s), key(s), and a touchscreen.

The user interface 910 functions to facilitate the capture of commands from the user such as an on-off commands or settings related to operation of the system described above. The user interface 910 may function to issue remote instantaneous instructions on images received via a non-local image capture mechanism.

A display 912 may also be operatively connected to the processor 920. The display 912 may include a screen or any other such presentation device that enables the user to view various options, parameters, and results. The display 912 may be a digital display such as an LED display. The user interface 910 and the display 912 may be integrated into a touch screen display.

The operation of the computing device 900 and the various elements and components described above will be understood by those skilled in the art with reference to the method and system according to the present disclosure.

It will be understood that while exemplary features of a distributed network system in accordance with the present teaching have been described that such an arrangement is not to be construed as limiting the invention to such features. The method of the present teaching may be implemented in software, firmware, hardware, or a combination thereof. In one mode, the method is implemented in software, as an executable program, and is executed by one or more special or general purpose digital computer(s), such as a personal computer (PC; IBM-compatible, Apple-compatible, or otherwise), personal digital assistant, workstation, minicomputer, or mainframe computer. The steps of the method may be implemented by a server or computer in which the software modules reside or partially reside. Generally, in terms of hardware architecture, such a computer will include, as will be well understood by the person skilled in the art, a processor, memory, and one or more input and/or output (I/O) devices (or peripherals) that are communicatively coupled via a local interface. The local interface can be, for example, but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the other computer components. The processor(s) may be programmed to perform the functions of the first, second, third and fourth modules as described above. The processor(s) is a hardware device for executing software, particularly software stored in memory. Processor(s) can be any custom made or commercially available processor, a central processing unit (CPU), an auxiliary processor among several processors associated with a computer, a semiconductor based microprocessor (in the form of a microchip or chip set), a macroprocessor, or generally any device for executing software instructions.

Memory is associated with processor(s) and can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory may incorporate electronic, magnetic, optical, and/or other types of storage media. Memory can have a distributed architecture where various components are situated remote from one another, but are still accessed by processor(s).

The software in memory may include one or more separate programs. The separate programs include ordered listings of executable instructions for implementing logical functions in order to implement the functions of the modules. In the example of heretofore described, the software in memory includes the one or more components of the method and is executable on a suitable operating system (O/S).

The present teaching may include components provided as a source program, executable program (object code), script, or any other entity including a set of instructions to be performed. When a source program, the program needs to be translated via a compiler, assembler, interpreter, or the like, which may or may not be included within the memory, so as to operate properly in connection with the O/S.

Furthermore, a methodology implemented according to the teaching may be expressed as (a) an object oriented programming language, which has classes of data and methods, or (b) a procedural programming language, which has routines, subroutines, and/or functions, for example but not limited to, C, C++, Pascal, Basic, Fortran, Cobol, Perl, Java, Json and Ada.

When the method is implemented in software, it should be noted that such software can be stored on any computer readable medium for use by or in connection with any computer related system or method. In the context of this teaching, a computer readable medium is an electronic, magnetic, optical, or other physical device or means that can contain or store a computer program for use by or in connection with a computer related system or method. Such an arrangement can be embodied in any computer-readable medium for use by or in connection with an instruction execution system, apparatus, or device, such as a computer-based system, processor-containing system, or other system that can fetch process the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be for example, but not limited to, an electronic, magnetic, optical, electromagnetic, infrared, or semiconductor system, apparatus, device, or propagation medium. Any process descriptions or blocks in the Figures should be understood as representing modules, segments, or portions of code which include one or more executable instructions for implementing specific logical functions or steps in the process, as would be understood by those having ordinary skill in the art.

It should be emphasized that the above-described embodiments of the present teaching, particularly, any “preferred” embodiments, are possible examples of implementations, merely set forth for a clear understanding of the principles. Many variations and modifications may be made to the above-described embodiment(s) without substantially departing from the spirit and principles of the present teaching. All such modifications are intended to be included herein within the scope of this disclosure and the present invention and protected by the following claims.

The invention is not limited to the embodiment(s) described herein but can be amended or modified without departing from the scope of the present invention, which is intended to be limited only by the scope of the appended claims as interpreted according to the principles of patent law including the doctrine of equivalents.

1. A system for determining one or more characteristics of a user based on an image of the user's eye, said system comprising:
a headset having a camera configured to acquire an image of the user's eye;
a computing device communicatively coupled to said camera and configured to:
receive the image of the user's eye; and
determine one or more characteristics of the user based on the received image.

2. The system of claim 1, wherein said headset comprises:
a substantially helmet-like headset that is configured to encapsulate at least a portion of the user's head; or
a pair of glasses.

3. The system of claim 1, wherein the one or more determined characteristics include one or more of: the age of the user, identity of the user, gender of the user, one or more health characteristics of the user.

4. The system claim 1, wherein said headset comprises an augmented reality or virtual reality headset.

5. The system of claim 1, wherein the image of the user's eye comprises an image of the user's retina.

6. The system of claim 5, wherein the image of the user's retina includes the Optic Nerve Head (ONH) and surrounding area.

7. The system of claim 1, wherein said computing device is further configured to provide the one or more determined characteristics to the user.

8. The system of claim 7,
wherein said headset comprises a display configured to visually display the one or more determined characteristics to the user; and/or
wherein said computing device comprises a display configured to visually display the one or more determined characteristics to the user.

9. The system of claim 1, wherein said computing device is configured to acquire a plurality of images of the user's eyes at predetermined intervals.

10. The system of claim 9, wherein said computing device is configured to compare the determined characteristics of the user across the plurality of images and alert the user to one or more changes in the one or more determined characteristics over a period of time.

11. The system of claim 1, wherein to determine the one or more characteristics of the user based on the received image, said computing device is configured to:
segment the image of the user's eye into multiple segments each containing blood vessels and neuroretinal rim fibres;
extract features from the segmented images, the extracted features including elements of the eye that intersect with the superimposed concentric geometric patterns, the extracted features describing relationships between the blood vessels themselves and between the blood vessels and the neuroretinal rim fibres in each of the segmented images; and
identify characteristics of the eye based on the extracted features.

12. The system of claim 11, wherein said computing device is configured to superimpose multiple concentric geometric patterns on the multiple segments.

13. The system of claim 12, wherein, the geometric patterns comprise concentric circles, ellipses, squares, or triangles.

14. The system of claim 11, wherein said computing device is further configured to classify the image of the eye based on the identified characteristics.

15. A method for determining one or more characteristics of a user based on an image of the user's eye, said method comprising:
providing a user with a headset comprising a camera;
acquiring an image of the user's eye using the camera;
transmitting the acquired image of the user's eye to a computing device that is communicatively coupled to the camera; and
determining one or more characteristics of the user based on the acquired image.

16. The method of claim 15, wherein said determining one or more characteristics of the user based on the received image comprises:
segmenting the image of the user's eye into multiple segments each containing blood vessels and neuroretinal rim fibres;
extracting features from the segmented images, the extracted features describing relationships between the blood vessels themselves and between the blood vessels and the neuroretinal rim fibres in each of the segmented images; and
identifying characteristics of the eye based on the extracted features.

17. The method of claim 16, further comprising superimposing multiple concentric geometric patterns on the multiple segments.

18. The method of claim 17, wherein the geometric patterns comprise concentric circles, ellipses, squares, or triangles.

19. The method of claim 15, further comprising:
acquiring a plurality of images of the user's eyes at predetermined intervals;
comparing the determined characteristics of the user across the plurality of images; and
alerting the user to one or more changes in their determined characteristics over a period of time.

20. Use of a headset for determining one or more characteristics of a user based on an image of their eye using the method as recited in claim 15.
